(19) United States
(12) Patent Application Publication
(10) Pub. No.: US 2002/0103091 A1
(43) Pub. Date: Aug. 1, 2002

(54) Reactive oil compositions and uses thereof

(76) Inventor: Dharma R. Kodali, Plymouth, MN (US)

Correspondence Address: Charles G. Carter, FOLEY & LARDNER, Firstar Center, 777 East Wisconsin Avenue, Milwaukee, WI, 53202-5367, US

(21) Appl. No.: 09/772,192

(22) Filed: Jan. 29, 2001

(51) Int. Cl. C07H 13/02; C10M 101/00; C07C 69/587

(52) U.S. Cl. 508/485; 536/119; 554/223

(57) ABSTRACT

A process for modifying an unsaturated polyol fatty acid ester stock, such as an unsaturated triacylglycerol oil, to enhance its reactivity provided. The method includes reacting the unsaturated polyol fatty acid ester stock with an oxygenating agent, such as an oxygen-containing gas. Tempering oils containing reactive polyol fatty acid esters and methods for their production and use are also provided.

### BACKGROUND

A wide variety of coated and/or treated composite substrate are used as construction materials. The composite structures are typically formed by heating and compressing a mat of fiber and/or particles impregnated with a resin binder. The mat may be treated, either before and/or after pressing, with agents such as release agents, tempering oils and decorative coating, to facilitate processing of the composite and/or alter its physical and visual properties. While the most common fiber/particle components for such composites are cellulosic, such as wood particles, fibers, flakes, chips and/or or other wood fragments, there has also been significant research and development directed toward use of fibers/particles from other sources such as glass, synthetic polymers, carbon and inorganic fillers (e.g., talc, alumina, silica, calcium carbonate and fly ash). As used herein, “wood composition board” refers to cellulose based composite substrates and includes the various hardboards, fiber boards, particle boards, wafer boards, strand boards and other related cellulose based composites, such as wet processed hardboards, dry processed hardboard, wet/dry processed hard boards, medium density fiber board, and oriented strand board.

Composite substrates are typically fabricated from a mixture of particles, fibers, flakes or chips with a binder, typically a thermosetting resin. The mixture is formed into a mat under wet-dry or dry process conditions and then compressed under heat and pressure into a dense composite substrate, typically in a sheet form. In some applications, such as in the manufacture of door skins, the mat may be molded into a desired shape and/or provided with a smooth or textured surface during the thermal compression process.

Fiberboards (also referred to as pressboards or hardboards) are boards manufactured from fibers interfelted, typically with a binding material, to produce an initial adhesive bond among the fibers. In a common procedure, the wet interfelted panels are shaped (e.g., in a heated metal press) and cut to the approximate desired dimensions (ordinarily to form a semi-hard board) and transferred to a drying and baking oven wherein the remaining moisture is evaporated and the boards are baked to set the binders. To allow the fiber panel to be released from the platen and the press for subsequent compression to size, the panel may be treated prior to drying and compression with a thermoset resin blend. An additional treatment of the basic board to enhance water resistance (tempering) is often carried out with various combinations of polymeric materials and/or drying oils. Commonly, the compressed board is immersed while still hot in a bath of a curable material, such as a drying oil, so that the surface and edges of the board become impregnated with the oil. The curable material may also be applied by spraying or roll coating. The impregnated board is then typically baked at high temperature to cure (polymerize) the curable material to a tough, insoluble form to yield the tempered fiberboard. The tempered fiberboard generally exhibits substantially improved physical properties, such as strength, hardness, and resistance to moisture, relative to untempered fiberboard.

In a common method of producing hardboard, wood logs or billets are reduced to chips. The chips are subjected to a cooking treatment with steam to soften them, then reduced to fibers by a mechanical refining process. Alternatively, increasingly high steam pressures may be used on the chips, followed by a quick depressurization to disintegrate the chips into fibers. The fibers can then be felted into fiberboard using either a wet process or a dry process. In the wet felting processes, the fibers are formed into an aqueous slurry of suitable consistency which is chemically treated as with binding oils or resins, pH adjusting chemicals and the like and the slurry is passed onto the drainage wire of a forming machine to produce a wet mat by drainage of the aqueous suspension. The mat may then be wet pressed or dry pressed, but generally the cold pressed wet mat is pressed against a wire screen and partially dried, e.g., by passing the mat into hot air dryers to reduce the moisture content to a relatively low value (e.g. less than 1%). The dried mat is commonly then hot pressed at high temperature (e.g., 400°-600° F.) under high pressure (e.g., 500-1,000 p.s.i.) for a short period of time; oven baked and rehumidified in humidifying chambers. The resulting product may constitute the final product for industrial uses or it may be further fabricated and decoratively finished, e.g., by applying various coatings and/or cutting on the surface for a decorative patterned hardboard. Dry fiber felted hardboard follows the same general process sequence as above except the fiber furnish is dried after preparation from the wood chips and before chemical treatment and mat formation. Fiber handling and mat forming techniques, of course, differ from wet process methods in that the fiber is handled in air and not in water.

Fiberboard produced by the processes described above, while possessing many excellent properties, can suffer from low strengths and high moisture take-up. Untreated fiberboard can absorb water which generally reduces the strength of the board and frequently causes appreciable dimensional changes in the board. The attendant swelling and shrinkage causes destruction of the boards at their edges, reduces racking strength and may result in usage disformation. In the past such problems have generally been addressed by “tempering/baking” standard fiberboard. Historically, oil tempered boards have been manufactured by dipping the pressed fiberboard in a large tank of drying oil, leaving the fiberboard for a short interval of time in the oil to permit absorption, and thereafter baking the oil soaked board in an oven at a temperature of about 300°-450° F. for several hours to cure the oil into a resin-like material. Instead of being applied via a dipping process, the oil can be applied by conventional roller coating or spraying techniques. Using such methods, an additional 1% to 3% by dry weight of the board of tempering oil is typically absorbed into and on the fiberboard and then cured by high temperature baking.

After the application of tempering oil, the treated fiberboards are commonly stacked in a rack and heated in an oven at about 300° F. for several hours to cure the tempering oil and further bind the fibers. During this baking period, however, the treated fiberboards generally produce volatile organic compounds (VOCs) that can be released and pollute the environment. VOCs produced in a manufacturing plant must be closely monitored and often removed via expensive pollution control devices.

It would be very advantageous to have tempering oils that could be applied and cured without releasing high levels of VOCs. This could likely be achieved if a tempering oil were available that could impart the desired properties to composite substrates, such as fiberboard, without the need for a curing step which includes the high temperature baking process. To provide such a capability, a tempering oil should be reactive enough to cure without extensive high temperature baking while be sufficiently fluid to enable penetration into the surface(s) of a pressed composite material. cl SUMMARY

The present invention relates to modified unsaturated fatty acid ester-based stocks which may be useful in producing materials such as tempering oils. The modified unsaturated fatty acid ester-based stocks may include modified polyol fatty acid ester unsaturated stocks, such as modified unsaturated triacylglycerol oils. The present application particularly concerns modifications of selected vegetable oils to produce reactive products with advantageous properties for use as reactive treating agents, for example as tempering oils, or in related applications. The unsaturated triacylglycerol polyol fatty acid ester stocks are commonly derived from plant sources, such as an oil seed, or animal sources, such as fish oils.

The modified unsaturated fatty acid ester-based stocks are reactive materials that may useful as tempering oils for treating composite substrates. The reactive materials are typically drying or semidrying oils with very high peroxide values (PVs) and sufficiently low viscosity to allow effective penetration into surfaces of composite substrates. As used herein, “drying oil” refers to an oil which, when applied as a thin film, is capable of absorbing oxygen from the air and polymerizing to form a tough, elastic film. Examples of drying and semidrying oils include linseed oil, soybean oil, safflower oil, dehydrated castor oil, tall oil, tung oil, oiticica oil, isano oil and/or menhaden oil. “Drying oil” commonly is used to refer to oils with an Iodine Value>140. As used herein, “semidrying oil” refers to oils with an Iodine Value of 120-140.

The present reactive materials are useful as tempering oils which can be applied on cellulose based fiberboard surfaces (or the surfaces of other related composite substrates) before or after the high temperature compression step. In a particularly useful embodiment, the reactive materials may be applied as a tempering oil to compressed fiberboard as the board is removed from a high temperature press and before board has cooled significantly. At this stage of the manufacturing process, the compressed fiberboard may still be at a temperature of 250-350° F. (circa 120-175° C.). Although the compressed fiberboard does not commonly remain at such a temperature for more than a few minutes after exiting the hot press, immediate application (e.g., within 1 to 2 minutes) can facilitate the initiation of the curing process. During this short time period after exiting the press, there is still typically sufficient retained heat in the compressed board to maintain the temperature of the board at about 210° F. (circa 100° C.)) or above. The application of the tempering oil while the compressed board is still at such a temperature, permits the remainder of curing of the tempering oil to be carried out at lower temperatures. For example, the remainder of the curing process may then be carried out while the compressed board is closer to ambient temperature (i.e., at a temperature of no more than about 50° C.). Alternatively, the tempering oil may be applied to the compressed board at a relatively low temperature. Under such circumstances, the curing process will generally require a longer period of time.

The process of curing a tempering oil typically requires oxygen, which is generally not available inside a closed hydraulic press. Since the present reactive tempering oils contain significant amounts of reactive oxygen atoms (in the form of peroxides), the reactive tempering oils can also be applied prior to the compression step. The high temperatures commonly employed in this step can initiate the decomposition of peroxide groups (e.g., through homolytic cleavage) and set off the curing process even before the compressed board has been removed from the press. After removal from the press, the curing process will generally continue, aided by oxygen available from the air and retained heat in the board.

A process for modifying an unsaturated polyol fatty acid ester stock, such as an unsaturated vegetable oil stock, to enhance its reactivity is also provided herein. The process includes reacting unsaturated polyol fatty acid ester stock with an oxygenating agent to form a blown ester product. Typical reaction conditions include reacting a drying or semidrying oil (e.g., linseed oil) with oxygen gas at a temperature of about 160-240° F. (circa 70-115° C.). The oxygenation of the unsaturated ester stock is commonly carried out by blowing a stream of an oxygen-containing gas, such as air, through an unsaturated ester stock which has already been heated to about 170-210° F. (circa 75-100° C.). It will be understood that the oxygenation of the starting ester stock will not necessarily be uniform, but rather may result in oxygenation of some fatty acyl chains and not of others. If desired, the blown ester product may be fractionated using conventional techniques to alter the spectrum of modified and unmodified esters present. For example, the blown ester product may be fractionated to remove at least a portion of the saturated esters, thereby altering the fluidity properties of the fractionated product with respect to the originally formed blown ester product.

The present method allows the production of reactive ester stocks which can have a peroxide value (“PV”) of at least about 150 Meq/Kg and a viscosity of no more than about 1,000 cPs. Unless otherwise designated, all viscosities referred to herein refer to viscosities determined using a Brookfield viscometer at 25° C. Certain embodiments of the method allow the production of reactive ester stocks with significantly higher peroxide values, e.g., reactive ester stocks with a PV of at least about 200 Meq/Kg, more desirably at least about 300 Meq/Kg and in some instances with a PV of 500 Meq/Kg or higher. The process also allows the production of ester stocks with high peroxide values and relatively low viscosity, e.g., a viscosity of no more than about 250 cPs and more desirably no more than about 100 cPs.

The term “unsaturated polyol fatty acid ester stock” as used herein refers to fatty acid esters of alcohols which include two or more hydroxy groups (“polyols”), where at least some and, typically, a majority (i.e., more than 50%) of the fatty acyl chains include at least one unsaturated carbon-carbon bond. The unsaturated polyol fatty acid esters employed in the present process typically include esters of a mixture of fatty acids, e.g., a mixture of saturated and unsaturated fatty acids. The mixture of fatty acids isolated from complete hydrolysis of a specific unsaturated polyol fatty acid ester stock is referred to herein as the “fatty acid composition” corresponding to that particular ester stock. In other words, by the term “fatty acid composition” reference is made to the identifiable fatty acids derived from the fatty acyl residues in the various polyol esters in a given stock. The distribution of specific identifiable fatty acids is typically characterized by the amounts of the individual fatty acids as a weight percent of the total mixture of fatty acids obtained from hydrolysis of the particular oil stock.

The term “conjugatively unsaturated polyol fatty acid ester stock” as used herein refers to polyol fatty acid ester(s) where at least some and, typically, a majority (i.e., more than 50%) of the fatty acyl chains include at least two unsaturated carbon-carbon bonds which are in conjugation. Examples of conjugatively unsaturated polyol fatty acid ester stocks include tung oil, oiticica oil and isano oil. Tung oil typically contains 70-90% eleostearic acid (a 9,11,13-18:3 fatty acid) as well as significant amounts of oleic acid (5-10%) and linoleic acid (5-15%). Oiticica oil commonly contains 50-80% of a mixture of 18:3 conjugated fatty acid and 18:3 conjugated keto fatty acid. Isano oil typically includes a substantial amount (e.g., 60-80%) conjugated diynoic fatty acids. Conjugatively unsaturated polyol fatty acid ester stocks can also be produced from unsaturated polyol fatty acid ester stocks (e.g., soybean oil, safflower oil, sunflower oil, castor oil, tall oil, oiticica oil, and/or menhaden oil) by the process described in U.S. Pat. No. 5,719,301, the disclosure of which is herein incorporated by reference.

The amount of conjugatively unsaturated groups in a polyol ester stock can be characterized based on the average number of conjugated polyunsaturated groups per fatty acyl chain (referred to herein as the “average conjugated unsaturation content”). In other words, the “average conjugated unsaturation content” of a polyol ester stock is the total number of conjugated systems divided by the total number of fattyacyl chains. As referred to herein, each conjugated system in a fatty aid chain (e.g., each conjugated diene, conjugated triene, or conjugated diyne) is counted as one conjugatively unsaturated group. Examples of vegetable oils with high average conjugated unsaturation contents include tung oil, isano oil (ongokea oil) and oiticica oil. For example, tung oil typically has a fatty acid composition which includes about 80% of a fatty acid which contains a conjugated triene group spanning carbons 9-14 (i.e., a 9,11,13-18:3 fatty acid). In this example, if all the fatty acids in the tung oil had the same chain length, the average conjugated unsaturation content of the tung oil would be 0.8. In actual practice, oils are generally made up of a mixture of fatty acids with some variation in chain length. Correspondingly, an unsaturated polyol ester stock with an average conjugated unsaturation content of 0.05 would refer to an oil stock in which, on average, 5% of the fatty acyl chains contain a single conjugated system (conjugated diene, triene, tetraene, diyne or the like).

The average chain length of the fatty acids can be calculated for a given fatty acid composition. Together with a determination of the total number of conjugatedly unsaturated groups (e.g., conjugated dienes, conjugated trienes, conjugated diynes) in the oil, this permits a calculation of the average conjugated unsaturation content for the oil. The total amount of conjugatedly unsaturated groups in a given unsaturated polyol ester stock can be determined by conventional techniques known to those of skill in the art, e.g., via absorption and/or nuclear magnetic resonance spectroscopy. Since saturated and nonconjugated unsaturated fatty acids exhibit only weak absorption in the UV region (between 200 and 400 nm) of the spectrum, UV absorption methods can be used to quantitate the amounts of conjugated polyunsaturated components present in a sample. FIG. 1 depicts a typical ultraviolet absorption spectrum for tung oil between 220 and 310 nm showing the absorption curve for an oil rich in conjugated trienoic esters of 9,11,13-octadecatrienoic acid (eleostearic acid) with absorption maxima at 260, 270 and 280 nm. Other conjugated polyunsaturated chromophores have strong absorption maxima in the UV region. For example, conjugated dienes such as 9,11-octadecadienoic acids and esters show a strong absorption band at around 230-235 nm and conjugated tetraenes show intense absorption closer to 400 nm.

The polyol fatty acid ester stocks subjected to reaction with an oxygenating agent in the present method may have quite low average conjugated unsaturation contents. In order to enhance the reactivity of the oil, e.g., for use as a tempering oil, the oxygenated blown oil product can be blended with a second polyol ester stock which has a relatively high average conjugated unsaturation content oil (such as tung oil and/or dehydrated castor oil).

Herein, when reference is made to the terms “unsaturated triacylglycerol oil” or “unsaturated triacylglycerol stock,” the intent is to refer to a material comprising triacylglycerols, at least a portion of which include unsaturated carbon-carbon double bonds. Very often, the triacyglycerols, whether altered or not, are derived from various plant and animal sources, such as oil seed sources. Unsaturated polyol fatty acid ester stocks, such as an unsaturated triacylglycerol stock, may also be produced synthetically, e.g., via a reaction between glycerol with fatty acid and/or fatty acid alkyl esters at least a portion of which include unsaturated fatty acyl chains. The term unsaturated triacylglycerol stock at least includes within its scope: (a) such materials which have not been altered after isolation; (b) materials which have been refined, bleached and/or deodorized after isolation; (c) materials obtained by a process which includes fractionation of an unsaturated triacylglycerol oil; and, also, (d) oils obtained from plant or animal sources and altered in some manner, for example through chemical modification by treatment with a metallic catalyst.

It will be understood that unsaturated polyol fatty acid ester stock can include a mixture of polyols, and a mixture of unsaturated polyol ester isomers. For example, unsaturated triacylglycerol oils typically include a mixture of triacylglycerols, and a mixture of triacylglycerol isomers. By the term “unsaturated polyol ester isomers” (as well as “unsaturated polyol fatty acid ester isomers”), reference is meant to polyol esters which, although including the same esterified fatty acid residues, may vary with respect to the location of the residues in the polyol ester. For example, an unsaturated triacylglycerol oil such as a vegetable oil stock can include both symmetrical and unsymmetrical isomers of a triacylglycerol which includes two or three different fatty acyl chains (e.g., includes both stearate and oleate groups). This can include triacylglycerol isomers that contain trans as well cis unsaturation and combinations thereof.

Herein, the result of reacting an oxygenating agent with an unsaturated polyol fatty acid ester stock, such as vegetable oil stock, will be referenced as an “blown ester product.” The term “blown ester product” includes within its scope the product of reacting one or more oxygenating agents (i.e., reagent(s) capable of adding one or more oxygen atoms to a fatty acid chain) with an unsaturated polyol fatty acid ester stock. As noted above, the individual polyol fatty acid esters which include at least one unsaturated carbon-carbon bond are referred to herein as unsaturated polyol fatty acid esters. As used herein, the terms “oxygenated chain” or “oxygenated group” refer to a reaction product resulting from the reaction of an oxygenating agent with a site on a fatty acyl chain (typically with an activated methylene group adjacent to an unsaturated carbon-carbon bond), such as in a fatty acyl chain of a triacylglycerol, to add one or more oxygen atoms to the moiety. One example of a blown ester product is the product resulting from reaction between oxygen (commonly introduced as air) and a highly unsaturated vegetable oil such as linseed oil.

Tempering oils which include reactive oxygenated fatty acid esters are also provided herein. The present reactive oxygenated fatty acid esters used as tempering oils commonly have a peroxide value of at least about 150 Meq/Kg, an average conjugated unsaturation content of at least about 0.02, and a viscosity of no more than about 1,000 cPs. More desirably, the reactive oils a peroxide value of at least about 250 Meq/Kg, an average conjugated unsaturation content of at least about 0.05, and/or a viscosity of no more than about 100 cPs. The present reactive oils typically have acid values of no more than about 3.0 mgKOH/g and densities on the order of 0.935 to 0.955.

Throughout this application, the text refers to various embodiments of the present compositions and/or related methods. The various embodiments described are meant to provide a variety illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### BRIEF DESCRIPTION OF THE DRAWINGS

FIG. 1 shows a typical ultraviolet absorption spectrum for tung oil between 220 and 310 nm.

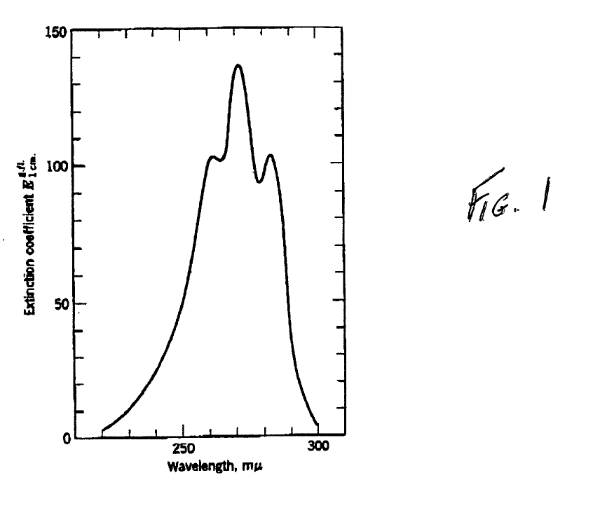

FIG. 2 shows a graph of the peroxide value and viscosity versus time for a sample of linseed oil reacted with oxygen according to the procedure described in Example 1.

FIG. 3 shows a graph of peroxide value and acid value versus time for a sample of linseed oil reacted with oxygen according to the procedure described in Example 1.

FIG. 4 shows a graph of peroxide value and density versus time for a sample of linseed oil reacted with oxygen according to the procedure described in Example 1.

FIG. 5 shows a graph of the peroxide value and viscosity versus time for a sample of linseed oil reacted with oxygen according to the procedure described in Example 1.

FIG. 6 shows a graph of peroxide value and acid value versus time for a sample of linseed oil reacted with oxygen according to the procedure described in Example 1.

FIG. 7 shows a graph of peroxide value and density versus time for a sample of linseed oil reacted with oxygen according to the procedure described in Example 1.

FIG. 8 shows a graph of peak maximum oxidation time (as determined by DSC) for moderate PV linseed oil (PV100 LSO) and a relatively high PV linseed oil (PV300 LSO) blended with varying amounts of tung oil.

### DETAILED DESCRIPTION

The present method may be utilized to oxygenate unsaturated fatty acid ester stocks, such as unsaturated triacylglycerol oils. For example, the method allows the production of vegetable oil-based reactive materials which, in addition to possessing very attractive reactive properties, are sufficiently fluid to be capable of penetrating the surfaces of composite substrates, such as wood fiber based materials (e.g., hardboard and medium weight fiber board). Since the reactive materials are typically derived from natural materials, these materials can be produced from available from renewable resources and generally have attractive raw materials costs.

I. Unsaturated Polyol Fatty Acid Esters

Unsaturated polyol fatty acid ester stocks include one or more unsaturated fatty acid esters and may also include one or more saturated fatty acid esters. Typically, the unsaturated fatty acid ester stocks used in the present method include a mixture of esters of saturated and unsaturated fatty acids, e.g., a mixture of saturated and unsaturated triacylglycerols, such as found in linseed oils and soybean oils. The polyol ester stocks are made up of fatty acid esters of alcohols which include two or more hydroxy groups (“polyols” or “polyhydric alcohols”). Fatty acid esters which are formed from polyhydric alcohols typically have all of the hydroxy groups esterified, although the present esters may have one or more unesterified hydroxyl groups. Such esters typically include at least two fatty acyl chains. Esters in which a portion of the hydroxy groups are esterified with a short chain carboxylic acid (i.e., carboxylic acids such as acetic and propionic acid which contain no more than 5 carbon atoms) are also included within the present definition of fatty acid esters. Typically no more than about 5 wt. % of the corresponding fatty acid composition and preferably much less is made up of short chain carboxylic acid(s). Desirably, at least all but one and, more preferably, all of the hydroxy groups of the alcohol portion of the esters are esterified to fatty acyl groups. As employed herein, the term “fatty acyl group” refers to an acyl group (“—C(O)R”) which includes an aliphatic chain (linear or branched) typically having from 6 to about 30 carbon atoms.

Polyols which can be used to form the present unsaturated fatty acid esters include at least two and, preferably, at least three hydroxy groups per molecule. Typically, the polyols have no more than 6 hydroxy groups per alcohol molecule and include up to about 20 carbon atoms and more commonly no more than 8 carbon atoms. Examples of suitable aliphatic polyols include glycerol, alkylene glycols (e.g., ethylene glycol, diethylene glycol, triethylene glycol and neopentylglycol), pentaerythritol, trimethylolethane, trimethylolpropane, and sorbitol. Suitable alicyclic polyols include cyclohexanediols and inositol as well as natural cyclic polyols such as glucose, galactose and sorbose.

Fatty acid esters of polyols which include no more than about 6 carbon atoms and have three to six hydroxy groups per molecule, such as glycerol, pentaerythritol, trimethylolethane, trimethylolpropane, sorbitol, inositol, glucose, galactose, and/or sorbose, are particularly suitable for use in the present invention. Commonly, such esters have a fatty acid composition which includes at least about 90 wt. % fatty acyl groups with aliphatic chains having from about 16 to 24 carbon atoms. The starting unsaturated fatty acid ester stocks employed to produce the present reactive esters typically have an Iodine Value of at least about 120, desirably at least about 150 and, more desirably, at least about 170.

II. Properties of Unsaturated Triacylglycerol Oils

Unsaturated triacylglycerol oils include triacylglycerol molecules (sometimes termed triglycerides). In general, triacylglycerols comprise three long fatty acid chains esterified to glycerol; or, alternatively phrased, glycerol esterified by addition thereto of three long chain fatty acids. Herein, the terms “triacylglycerols” and “triglycerides” are intended to be interchangeable, and will in some instances be referred to by the abbreviation “TAG”.

As indicated above, any given triacylglycerol molecule generally includes glycerol esterified with three fatty acid molecules. Thus, each triacylglycerol includes three fatty acid residues. In general, oils extracted from any given plant or animal source comprise a mixture of triacylglycerols characteristic of the specific source. The mixture of fatty acids isolated from complete hydrolysis of the triacylglycerols in a specific sample are referred herein to as the “fatty acid composition” of that oil. By the term “fatty acid composition” reference is made to the identifiable fatty acid residues in the various triacylglycerols.

The average number of double bonds present per fatty acyl chain in an unsaturated polyol fatty acid ester is referred to herein as the “average unsaturation content.” For example, the average unsaturation content of an unsaturated triacylglycerol oil may be calculated based from the distribution of fatty acids in the mixture produced by hydrolysis of the triacylglycerols. The distribution of fatty acids in a particular oil may be readily determined by methods known to those skilled in the art.

Another measure for characterizing the average number of double bonds present in a polyol fatty acid ester stock (such as the triacylglycerol molecules of an unsaturated triacylglycerol oil) is its Iodine Value. As referred to herein, the Iodine Value of a polyol fatty acid ester or mixture of polyol fatty acid esters is determined by the Wijs method (A.O.C.S. Cd 1-25). The present method can be used to enhance the reactivity of unsaturated polyol fatty acid ester having a wide range of Iodine Values. Typically, however, the present methods employ as starting materials unsaturated polyol fatty acid ester stocks, such as unsaturated vegetable oil stocks, having an Iodine Value of at least about 120, more desirably at least about 150, and, polyol fatty acid ester stocks having an Iodine Value of at least about 170 are particularly suitable for use in producing reactive ester stocks which can be used as tempering oils. For example, linseed oil typically has an Iodine Value of about 180-190.

III. Modifications to Unsaturated Polyol Fatty Acid Ester Stocks; Use as Tempering Oils

Modification of unsaturated triacylglycerol oils through formation of an oxygenated product can increase the reactivity (e.g., as measured by the peroxide value) of an oil with respect to the corresponding unmodified vegetable oil. The present modified esters may be produced by reacting a oxygenating agent with at least a portion of the unsaturated fatty acid ester stock. The oxygenating agent is generally capable of reacting with the fatty acyl chains of the triacylglycerols to add peroxide groups (“—O—O—H”) to the chains. Depending on the nature of the reaction conditions, the peroxide groups may decompose leading to the formation of secondary reactions products, e.g., the formation of fatty acyl chain which include other oxygen-containing functional groups and/or reactions which can result in the cleavage of a fatty acyl chain.

To produce reactive polyol ester stocks which can be used as tempering oils, it is particularly desirable to carry out the present oxygenation reaction so that the decomposition of the initially formed peroxide groups is minimized. This can typically be achieved by reacting an unsaturated polyol fatty acid ester stock with oxygen at a temperature of no more than 120° C. to form the blown ester product. Generally, the oxygenation reaction is carried out by heating the starting unsaturated oil to a temperature of at least 150° F. (circa 65° C.) and maintaining the reaction temperature at 210° F. (circa 100° C.) or below during the course of the reaction. Reaction temperatures of 160-190° F. (circa 70-90° C.) have been found to be quite suitable.

It is important to recognize that in commercial practice of the techniques described herein, the techniques will typically be operated on mixtures of triacylglycerols either isolated as a plant or animal oil, e.g., by various oil seeds processing techniques, or resulting from alteration of such oils, for example by prior fractionation and/or chemical modification.

Herein, in connection with oxygenation of an unsaturated fatty acid ester stock, reference will in some instances be made to “at least partially oxygenating.” By this, it is meant that the starting unsaturated polyol ester stock is treated under appropriate conditions to add a peroxide group to at least some of fatty acid chains of the ester(s). In order to be considered “at least partially oxygenated” as the term is used herein, there should be an increase of at least about 100%, and more commonly at least about 300% in the peroxide value of the oxygenated product in comparison to the original ester stock (which typically has a peroxide value of no more than about 50 MeqKg).

From the above, it will be understood that the intent is to reference techniques that may be practiced on mixtures, without precise analysis of exact adduct and unreacted triacylglycerol presence in the mixture, but rather with a general understanding of overall oxygenation during modification. The intent, in general, is to obtain a stock of desirable property with respect to, inter alia, peroxide value and viscosity. Indeed, it is foreseen that in some applications blends may well be desirable, depending on the use to which the reactive ester stock is to be placed. The present method is particularly useful for producing reactive base stocks which include a substantial amount of a modified unsaturated triacylglycerol oil, e.g., a reactive ester stock including at least about 50 wt. % and, preferably, at least about 75 wt. % of the modified unsaturated triacylglycerol oil. By employing the present method, triacylglycerol oil-based stocks which have a combination of reactivity and viscosity properties suitable for a variety of applications may be produced. Preferred embodiments of the invention include such reactive oil stocks having an acid number of no more than about 3.0 mgKOH/g, preferably no more than about 2.5 mgKOH/g; and/or a density of no more than about 0.96. For example, the illustrative experiments described herein demonstrate that it is possible to produce a reactive unsaturated polyol ester stock, which has a peroxide value of at least 300 MeqKg, a viscosity of no more than 100 cPs (@25° C.), a density of no more than 0.945, and an acid number of no more than 1.5 mgKOH/g, from linseed oil using the present method.

Techniques according to the present invention, as will be understood from the experimental report below, are particularly suitable for generating of desirable reactive ester stocks from vegetable oils. In general, this is because of the particular level of unsaturation found in many vegetable oils, as well as the physical properties both of starting materials and the final adducts. In general, improvement is observed if the extent of oxygenation is such that sufficient peroxide groups to confer a peroxide value of at least 150 Meq/Kg on the unsaturated ester stock. This can readily be controlled by judicious choice of the starting vegetable oil stock, the type and amount of peroxidation agent employed and the reaction conditions. More broadly, improvement in the reactivity of an unsaturated polyol ester stock, such as a vegetable oil stock, can be produced through the addition of sufficient oxygen to form an ester product with a peroxide value of at least about 150 Meq/Kg which not greatly increasing the viscosity of the oil stock. It has been found that the oxidation reaction can be controlled to produce reactive ester stocks with peroxide values of at least about 250 while maintaining a viscosity of 250 cPs or below (more desirably no more than 100 cPs). In certain embodiments of the present method, it is possible to produce an ester product which has a relatively high peroxide value (e.g. 300 to 400 Meq/Kg or higher) while maintaining quite a low viscosity (e.g., no more than about 100 cPs (@25° C.).

Particularly suitable vegetable oils for use with the present method include highly unsaturated oils, e.g., oils which have a fatty acid composition containing substantial amounts of polyunsaturated oils such as linoleic acid (a 9,12-octadecadienoic acid) and/or linolenic acid (a 9,12,15- or 6,9,12-octadecatrienoic acid). Other vegetable oils which, it is foreseen, may be modified with techniques according to the present invention, include: sunflower oil, soybean oil, safflower oil and fish oils, such as menhaden oil. Generally, the oils used as raw materials in the present process have an Iodine Value of at least about 120. Variants of some of the other oils listed above are also known, e.g., high oleic and very high oleic sunflower, canola and soybean oils. As discussed herein, these vegetable oils may be employed in the present invention as isolated or in altered form, as well as with oil from a single source or mixtures of one or more of the types of oils (or altered forms thereof).

In some instances, e.g., where soybean oil is used as a starting material, it may be useful to react the unsaturated polyol fatty acid ester stock with the oxygen in the presence of a peroxidation catalyst to produce the blown ester product. Where it is employed, the peroxidation catalyst typically is a salt of a metal ion such as Cu(I), Fe(II) and/or Fe(III). One peroxidation catalyst which has been found to be useful is a mixture of FeCl₂ and FeCl₃ (e.g., in a about a 1:1 to 5:1 ratio). Typically, the process of reacting the unsaturated polyol fatty acid ester stock with the oxygen can be facilitated by the presence of about 1 to 20 ppm of the peroxidation catalyst (e.g., based on the amount of the unsaturated polyol fatty acid ester stock). For example, a unsaturated ester stock (such as a semidrying oil like soybean oil) can be reacted with oxygen in the presence of about 0.5 wt. % of a 3:1 mixture of an FeCl₂/FeCl₃ peroxidation catalyst to produce the blown ester product. As with other reaction conditions, where a peroxidation catalyst is employed, the amount of catalyst is chosen so as to minimize the amount of decomposition of the peroxide groups to the extent possible.

The reaction of an unsaturated ester stock with an oxygen source can also be facilitated by adding a reactive ester stock having a peroxide value of at least 200 to the unsaturated polyol fatty acid ester stock before introducing the oxygen. Where such an approach is employed, commonly at least about 0.1 wt. % (and more desirably at least about 0.5 wt. %) of the high peroxide reactive ester stock is added to the initial reaction mixture. If desired, both a peroxidation catalyst and an aliquot of high peroxide reactive ester stock can be added to the reaction mixture prior to the introduction of the oxygenating agent.

The reactive ester stocks produced by the present process can be used as tempering oils. In a particularly suitable embodiment, a reactive oil (such as the present blown ester stocks) can be blended with a conjugatively unsaturated polyol fatty acid ester stock (e.g., tung oil) to form a tempering oil with enhanced reactivity. Such tempering oils commonly have a peroxide value of at least about 150 Meq/Kg, an average conjugated unsaturation content of at least about 0.02, and a viscosity of no more than about 1,000 cPs. More desirably, the reactive oils a peroxide value of at least about 200 Meq/Kg, an average conjugated unsaturation content of at least about 0.05, and/or a viscosity of no more than about 100 cPs. In particularly suitable embodiments, a sufficient amount of a conjugatively unsaturated oil, such as tung oil or dehydrated castor oil, is blended with the high PV unsaturated oil to produce a tempering oil with an average conjugated unsaturation content of about 0.05 to 0.3 and, more desirably about 0.1 to 0.2. This can be done, for example, by blending an appropriate amount (e.g., 5 to 25 wt. %) tung oil with a high PV modified vegetable oil. The reactive oils which are useful as tempering oils generally have acid values of no more than about 3.0 mgKOH/g and/or a density of no more than about 0.96. More desirably, the present tempering oils have densities on the order of 0.935 to 0.955 and/or an acid value of no more than about 2.5 mgKOH/g. Even with their high peroxide values, the reactive oils which are useful as tempering oils typically have at least relatively moderate levels of overall unsaturated character (e.g., as measured by Iodine Value). For example, where a semidrying oil (such as soybean oil) is used as a raw material, the tempering oil generally has an Iodine Value of at least about 110 and typically higher. It is not uncommon for the present tempering oils to have a peroxide value of at least 150-200 Meq/Kg while retaining Iodine Values of at least 150 and, in some instances, 170 or higher.

The tempering oils suitably include at least about 80 wt. % and, more desirably, at least about 90 wt. % of the reactive polyol fatty acid ester stock. The tempering oils may also include other components to impart specific properties to the treated composite and/or to alter the physical properties of the tempering oil. For example, a solvent may be included in the tempering oil to lower its viscosity and enhance its ability to penetrate into surfaces of the composite substrate.

When the present reactive ester stocks are to be used as tempering oils, their reactivity can be enhanced by the addition of a metallic drier. To avoid decomposition of the peroxide groups in the reactive ester stock during storage, the drier is typically blended into the tempering oil just prior to use. As used herein, “metallic driers” refer to catalysts which are added to drying and/or semi-drying oils to increase the rate of oxidation and/or cross-linking reactions which occur during curing of the oils. Metallic driers commonly include salts of polyvalent metals (metals with a valence of two or greater), such as salts of cobalt, manganese, cerium, lead, chromium, iron, nickel, zirconium, calcium, cerium and zinc. The salts can include the anionic form of organic acids as a counterion. Examples of common metallic driers include metal naphthenates, octanoates (e.g., 2-ethylhexanoates) and/or resinates as well as polyvalent metal salts of fatty acids (e.g., linoleates and/or salts of tall oil fatty acids). Metallic driers typically include a mixture of metal species, e.g., mixtures of iron and manganese salts or mixtures of iron, cobalt and manganese salts. Cobalt salts are reported to be very active surface driers for air dried finishes. In contrast, manganese salts are reported to have intermediate activity but can give better “through dry” than cobalt salts.

Tempering oils formed from the present reactive ester stocks can be used to treat composite substrates, such as fiber board, to produce a treated composite board. The tempering oil desirably has a peroxide value of at least about 150; an average conjugated unsaturation content of at least about 0.02; and a viscosity of no more than about 250 cPs.

The tempering oils can be applied to composite substrates, e.g., cellulose based fiberboard, either before or after the high temperature compression step. The tempering oil is applied to at least one side of the compressed composite substrate and, more commonly, to both major surfaces of a composite board. In a particularly useful embodiment, the reactive materials are applied as a tempering oil to compressed fiberboard just as the board is removed from a high temperature press and before board has cooled significantly. In this embodiment, the compressed fiberboard is desirably still at a temperature of at least about 250-350° F. (circa 120-175° C.) when the tempering oil is applied. The treated composite substrate is then allowed to cool to ambient temperature during which time the curing of the tempering oil continues.

For example, a tempered fiber board can be formed by pressing a compressible mat, which includes a plurality of cellulosic fibers and a resin binder, between a pair of heated plates to form a compressed fiber board. The compressed fiber board is removed from between the heated plates and the tempering oil to at least one side of the compressed fiber board before the temperature of the board has dropped below about 100° C. The board is then typically allowed to cool over a period of several hours, with the final stages of the curing process occurring after the board has been cooled to close to ambient temperature, e.g., while the treated fiber board is at a temperature of no more than about 50° C. (circa 120° F.).

In a related embodiment, the tempering oil is applied to at least one side of a compressible mat, which includes a resin binder and a plurality of fibers, particles, flakes, and/or chips. The compressible mat is pressed between a pair of heated plates to form a compressed fiber board. The heat imparted from the plates (which are commonly are high temperatures of 400°-600° F.) to the mat can be sufficient to initiate the curing process, which then continues after the compressed mat has been removed from between the plates and is allowed to cool, e.g., on a storage rack.

The invention will be further described by reference to the following examples. These examples illustrate but do not limit the scope of the invention that has been set forth herein.

### EXAMPLE 1

### Production of PV300 LSO

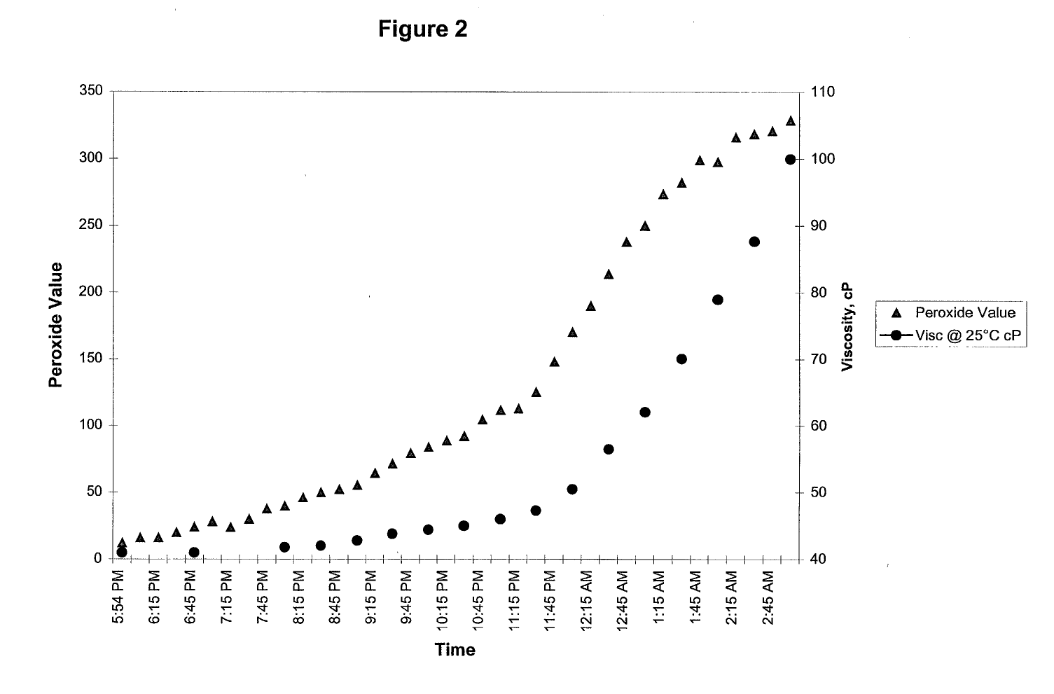

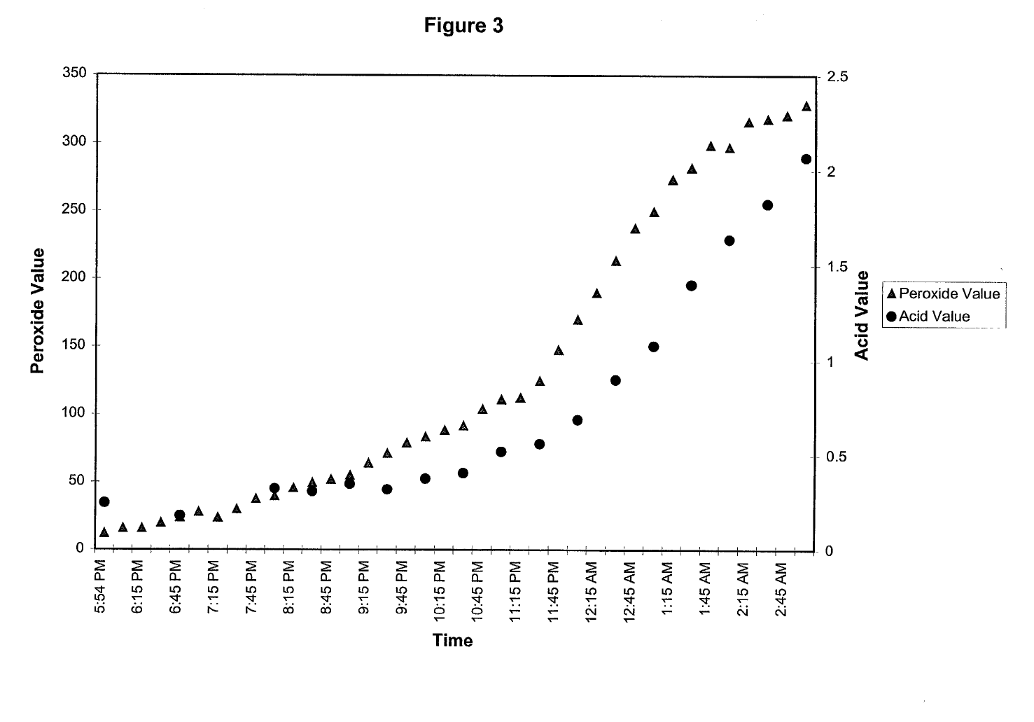

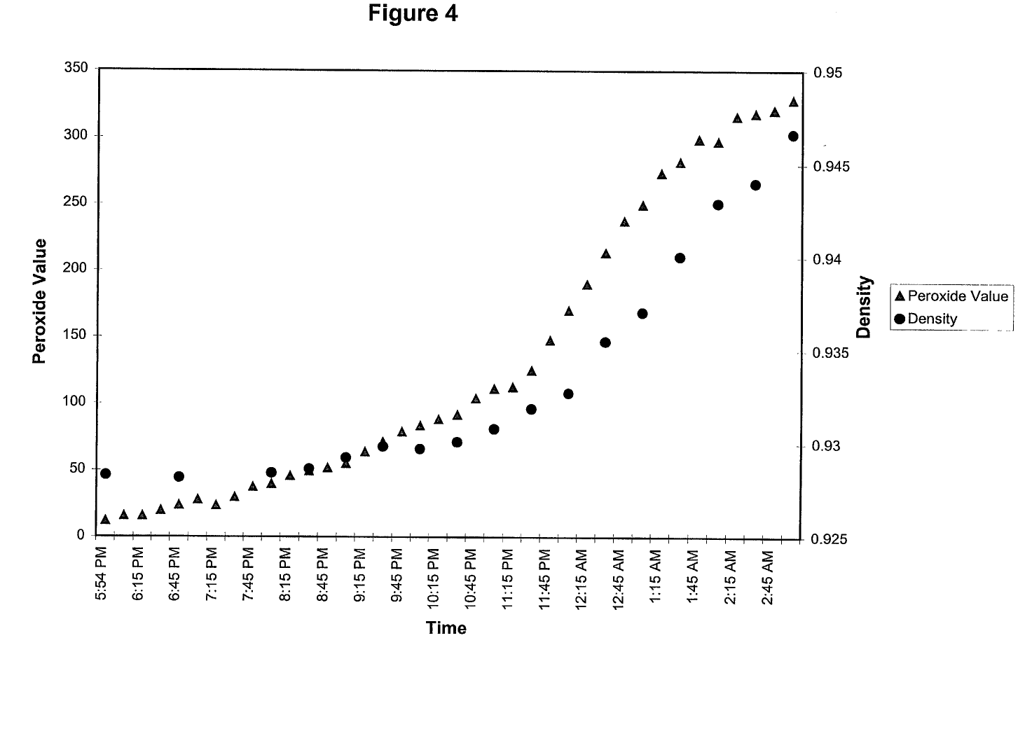

Linseed oil (45,000 lbs) was introduced into a reaction vessel. The linseed oil was a refined grade of linseed oil that has been processed by alkali refining, bleaching, chilling and filtering. The linseed oil in the reaction vessel was heated to a temperature of 195° F. (91° C.). Once the oil had been heated to this temperature, the air blower was started and operated such that approximately 2,500 cfm (cubic feet/minute) of air was passed through the oil. An aliquot removed from the reaction mixture shortly after initiation of the air flow showed that the oil at that point had a density of 0.928, a viscosity of 41 cPs (@25° C.), an acid value of 0.25 and a peroxide value of 12 Meq/Kg. After passing air through the oil for about 2 hours, the temperature of the reaction mixture had risen to about 200° F. (93° C.). The reaction mixture was maintained at this temperature for an additional 7.25 hours (9.25 hours total) while air was continued to be blown through the oil. FIG. 2 shows a graph of the peroxide value and viscosity of the reaction mixture as a function of time. FIG. 3 shows a graph of peroxide value and acid value of the reaction mixture as a function of time. FIG. 4 shows a graph of peroxide value and density of the reaction mixture as a function of time. The final product had a density of 0.947, a viscosity of 100 cPs (@25° C.), an acid value of 2.07 mgKOH/g, and a peroxide value of 329 Meq/Kg.

### EXAMPLE 2

### Production of High PV LSO Sample

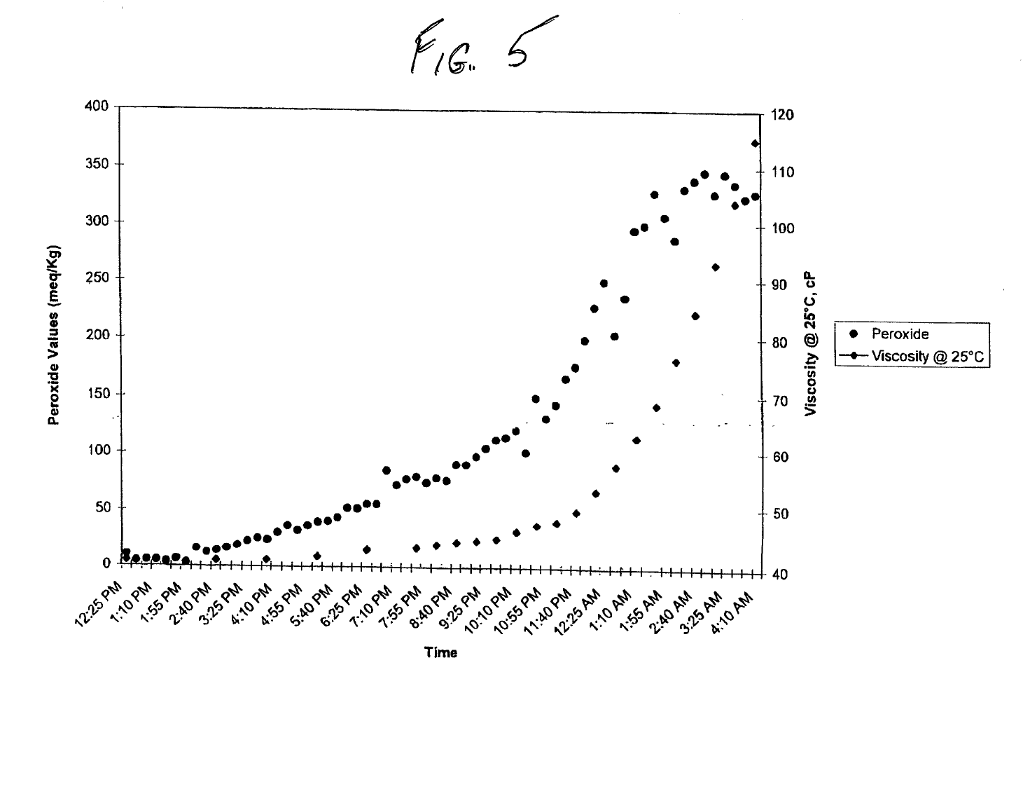

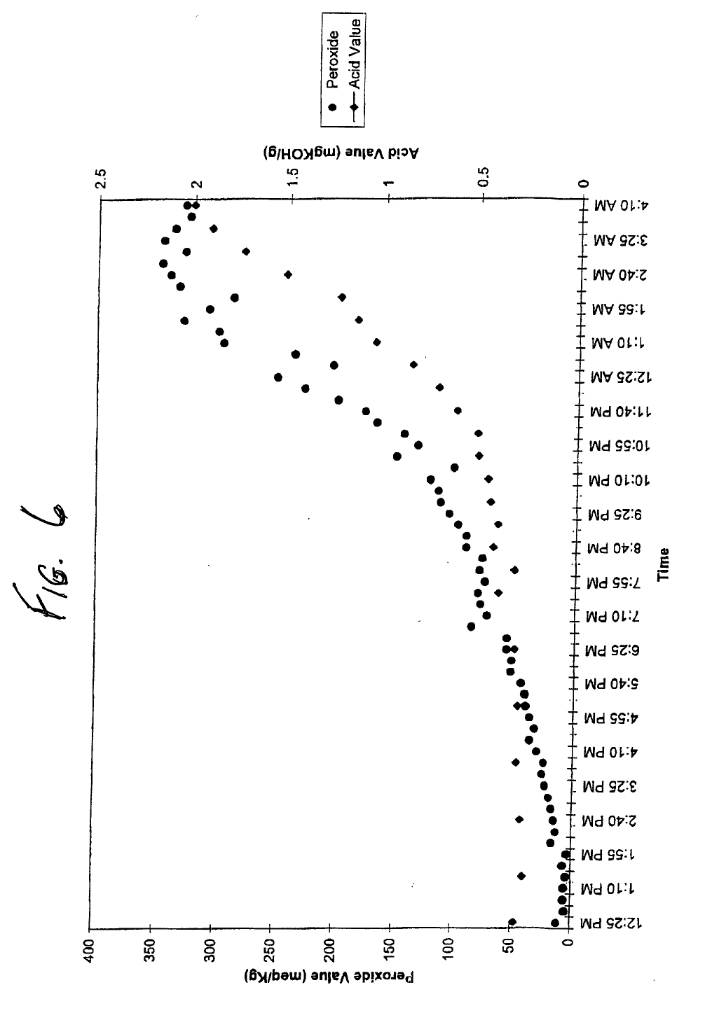

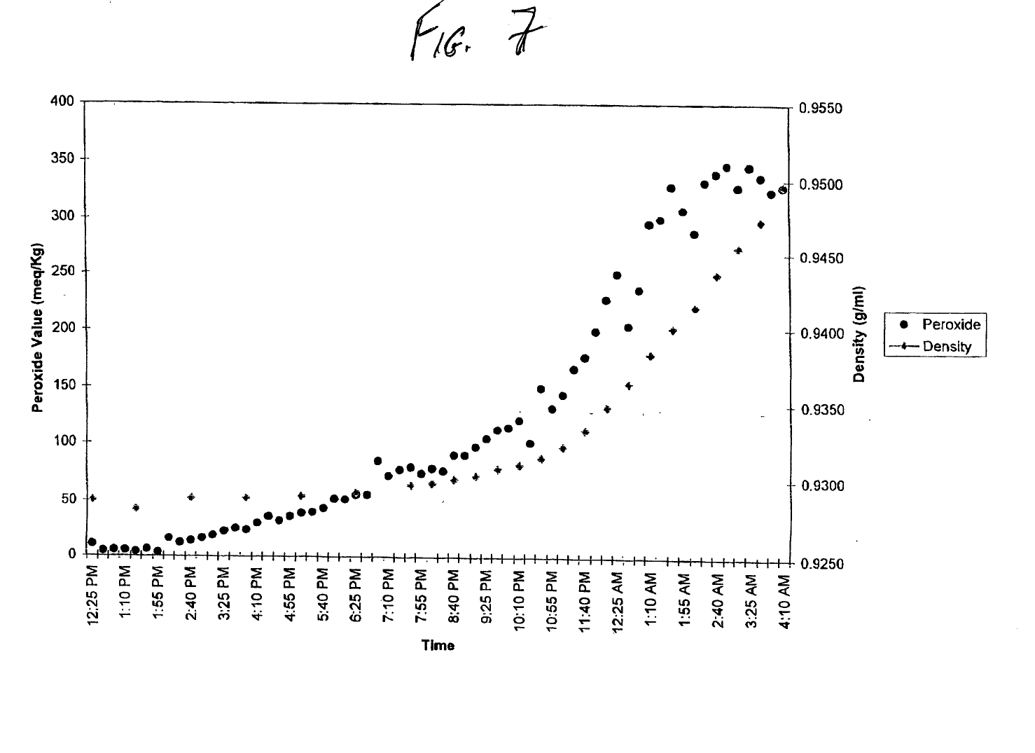

Linseed oil (45,000 lbs) was introduced into a reaction vessel. The linseed oil was a refined grade of linseed oil that has been processed by alkali refining, bleaching, chilling and filtering. The linseed oil in the reaction vessel was heated to a temperature of 179° F. (85° C.). Once the oil had been heated to this temperature, the air blower was started and operated such that approximately 2,500 cftn of air was passed through the oil. An aliquot removed from the reaction mixture shortly after initiation of the air flow showed that the initial oil had a density of 0.929, a viscosity of 41 cPs (@25° C.), an acid value of 0.29 mgKOH/g, and a peroxide value of 10.6 Meq/Kg. The while the temperature of the reaction mixture reaction mixture was maintained at a temperature of about 181 to 186° F. (circa 83 to 86° C.) for 14.5 hours while air was continuously blown through the oil. At this point, the reaction mixture had a density of 0.944, a viscosity of 84.4 cPs (@25° C.), an acid value of 1.7 mgKOH/g, and a peroxide value of 340 Meq/Kg. Air was blown through the reaction mixture for another 1.5 hours (16 hours total) while the temperature was maintained at about 185 to 188° F. (circa 85 to 87° C.). The final product had a density of 0.950, a viscosity of 115 cPs (@25° C.), an acid value of 2.0 mgKOH/g, and a peroxide value of 328 Meq/Kg. FIG. 5 shows a graph of the peroxide value and viscosity of the reaction mixture as a function of time. FIG. 6 shows a graph of peroxide value and acid value of the reaction mixture as a function of time. FIG. 7 shows a graph of peroxide value and density of the reaction mixture as a function of time.

### EXAMPLE 3

### Production of a Comparison Tempering Oil

Linseed oil (45,000 lbs; ASTM raw grade) was introduced into a reaction vessel. The linseed oil in the reaction vessel was heated to a temperature of 240° F. (circa 115° C.). Once the oil had been heated to this temperature, the air blower was started and operated such that approximately 2,500 cfm of air was passed through the oil for about 130 minutes. Once the air flow has been shut off, the reaction product was cooled to 180° F. (82° C.) or below and either pumped into a storage tank or transferred into containers suitable for shipment. The resulting combined aliquot of blown linseed oil had a density of 0.94-0.97, a Gardner-Holt viscosity of D (@25° C.), an acid value of ≦5 mgKOH/g, an Iodine Value of 150-170, and a saponification value of 195-205 (as determined by AOCS Cd 3-25).

A tempering oil was produced by blending 95 parts by weight of this blown linseed oil with 5 parts by weight tung oil. Retain samples from a number of batches of tempering oil produced by this procedure were pooled and analyzed. The sample of pooled aliquots had a density of 0.947, a viscosity of 112.5 cPs (@25° C.), an acid value of 2.49 mgKOH/g, and a peroxide value of 43.4 Meq/Kg. From a PDSC analysis of a sample from the pooled aliquots using the procedure described in Example 4 it was determined that the sample exhibited a peak maximum oxidation after 17.0 minutes (@ an initial temperature of 100° C. under 500 psi oxygen).

### EXAMPLE 4

### Oxidation Rates of High PV LSO/Tung Oils Blends

The relative reactivity of medium and high PV linseed oils blended with varying amounts of tung oil were examined using pressure differential scanning calorimetry (“PDSC”) on samples that had been heated to 100° C. under pure oxygen at 500 psi. The protocol was the same as that outlined in ASTM method D6186-98 except that the determination was carried out at 100° C. and peak maximum oxidation time was measured instead of an oxidation induction time (the samples were so reactive that essentially no induction time was observed). The peak oxidation times measured provide an indication of the relative reactivity of the samples and of their cure rates in the presence of an oxygen-containing atmosphere after initial being heated to 100° C. The medium PV linseed oil (PV100 LSO) was a sample of a lightly blown linseed oil with viscosity of less than 50 cPs (@25° C.) and a peroxide value of about 100 Meq/Kg. The high PV linseed oil (PV300 LSO) was a blown linseed oil produced by the present process with viscosity of circa 100 cPs (@25° C.) and a peroxide value of about 329 Meq/Kg.

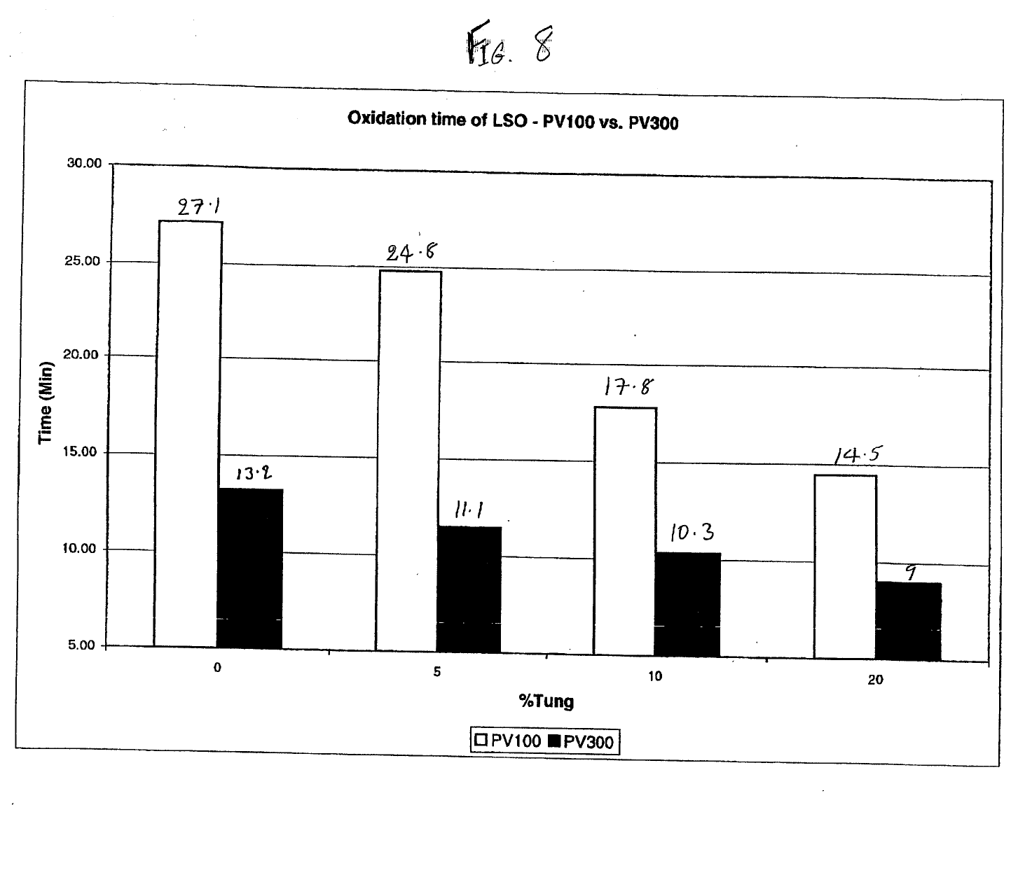

The results of the experiment are shown in FIG. 8. Either with or without the addition of a conjugated unsaturated oil such as tung oil, the high PV sample (PV300 LSO) undergoes considerably faster oxidation/curing as evidenced by the PDSC results. The peak oxidation time for the PV300 LSO sample was 13.2 minutes versus 27.1 minutes for the PV100 LSO sample. The inclusion of 5 wt. % tung oil has very little effect on the oxidation rate of the PV100 LSO sample. The peak oxidation time for the PV300 LSO sample is systemically decreased by the addition of 5, 10 and 20 wt. % tung oil. Moreover, pure PV300 LSO was determined to have a faster rate of air oxidation (as evidenced by a peak oxidation time of 13.2 minutes) than the medium PV LSO sample blended with up to 20 wt. % of a conjugated unsaturated oil, such as tung oil.

### EXAMPLE 5

### Production of a High PV Soybean Oil

Soybean oil (45,000 lbs) is introduced into a reaction vessel. A FeCl₂/FeCl₃ catalyst (10 ppm; 3/1 ratio) and 0.5 wt. % blown soybean oil (PV=300 Meq/Kg; viscosity=1.5 Stokes @25° C.) were added to the soybean oil. The reaction mixture is heated to a temperature of 200° F. (circa 95° C.). Once the oil has been heated to the desired starting temperature, the air blower is started and operated such that approximately 5,000 cftn of air is passed through the soybean oil for 10-15 hours while maintaining the reaction temperature at about 200° F. The blown soybean oil produced by this method will have a peroxide value of circa 350-450 Meq/Kg and a viscosity of no more than about 100 cPs @25° C.

### EXAMPLE 6

Linseed oil (45,000 lbs) is introduced into a reaction vessel. The linseed oil is typically an ASTM RAW grade linseed oil. Other grades of linseed oil that have been more highly processed and/or purified may also be used. For example, a refined grade of linseed oil that has been produced by alkali refining, bleaching, chilling and filtering an ASTM RAW grade linseed oil is quite suitable for use in the present process. The linseed oil in the reaction vessel is heated to a temperature of 180-200° F. (82-93° C.). Once the oil has been heated to the desired starting temperature, the air blower is started. The air blower is operated such that approximately 2500 cfm of air is passed through the oil. The temperature of the oil is monitored and maintained at a desired set temperature between 180° F. and 200° F. through the use of cooling coils submerged in the oil, by modulating the amount heat supplied to the oil, and/or by modulating the rate of air flow through the oil. The aliquots of the reaction mixture are removed at periodic time intervals and the progress of the reaction is monitored by determining the viscosity, peroxide value, density and/or acid value of the aliquots. The introduction of further air into the reaction mixture is generally stopped once the peroxide value of the mixture has reached a desired value or the rate of increase of the peroxide value falls below a specified rate. Alternatively, a desired set point for one or more other parameters (e.g., viscosity, acid value and/or density) can be used to determine the end point for the reaction. Once the air flow has been shut off, the reaction product is typically cooled to 180° F. or below and either pumped into a storage tank or transferred into containers suitable for shipment.

### EXAMPLE 7

A wood fiber mat impregnated with a binder resin is compressed between two heated plates of a conventional press at a temperature of about 350-400° F. for 1-2 minutes under a pressure of about 500-1,000 psi to form a compressed fiber substrate. At least one side of the compressed fiber substrate is coated with about 10-20 g/sq.ft. of a tempering oil while the temperature of the compressed fiber substrate is at least about 250° F. (the tempering oil is typically applied to the compressed fiber substrate within about 30-60 seconds after its removal from the press). The tempering oil is formed by blending 90 wt. % of a high PV blown linseed oil (e.g., PV=350; vicosity≦100 cPs) prepared according to the process described in Example 5 and 10 wt. % tung oil. Just prior to application to the wood fiber mat, 0.01-0.05 wt. % of a drier made up of Fe, Mn and Co carboxylate salts is added to the tempering oil. After application of the tempering oil, the compressed fiber substrate is allowed to cool to ambient temperature over a period of several hours. The resulting treated fiber composite board does not need to be subjected to a further baking step in order to complete the curing of the tempering oil.

### EXAMPLE 8

A wood fiber mat impregnated with a binder resin can be coated on at least one side with about 10-20 g/sq.ft. of a tempering oil before being pressed into a fiber board. The tempering oil is formed by blending 90 wt. % of a high PV blown linseed oil (e.g., PV=350; vicosity≧100 cPs) prepared according to the process described in Example 5 and 10 wt. % tung oil. Just prior to application to the wood fiber mat, 0.01-0.05 wt. % of a drier made up of Fe, Mn and Co carboxylate salts is added to the tempering oil. If desired, a release agent, such as a silicone release agent, can also be applied to the coated surface of the wood fiber mat after application of the tempering oil. The coated wood fiber mat is then compressed between two heated plates of a conventional press at a temperature of about 350-400° F. for 1-2 minutes under a pressure of about 500-1,000 psi. The resulting treated fiber composite board does not need to be subjected to a further baking step in order to complete the curing of the tempering oil.

### EXAMPLE 9

### Determination of the Peroxide Value of an Oil

The procedure emloyed is set forth in AOCS method Cd 8b-90. About 5 grams of an oil sample is weighed into a 125 ml Erlenmeyer flask and the accurate weight was recorded. To this sample was added 50 ml of a solvent mixture containing isooctane and glacial acetic acid in a ratio of 2/3 (v/v) and the resulting mixture was vigorously stirred at room temperature. Once the oil sample had been totally dissolved, 0.5 ml of saturated potassium iodide was added and the resulting mixture was stirred for 0.5 to 1 min. Distilled water (30 ml) and a pipette full of saturated starch solution were added sequentially. The blackish mixture was titrated with 0.1 N sodium thiosulfate solution until all the blackish color disappeared. The actual volume of the sodium thiosulfate consumed was recorded and the peroxide value (PV) was calculated using the following equation:

Peroxide Value (in Meq/Kg)=V×N×1000/W

Where

V=volume of the sodium thiosulfate solution used in the titration;

N=normality of sodium thiosulfate solution;

W=weight of the sample.

### EXAMPLE 10

### Determination of the Acid Value of an Oil

The Acid Value of oils were determined by the procedure described in AOCS method Cd 3a-63. About 2.5 grams of an oil sample is weighed into a 250 ml Erlenmeyer flask and the accurate weight was recorded. To this sample was added 125 ml of a solvent mixture containing toluene and isopropanol in a ratio of 1/1 (v/v) and the resulting mixture was vigorously stirred at room temperature. Once the oil sample had been totally dissolved, 2 ml of 1.0% phenolphthalein in ethanol was added and the resulting mixture was stirred for 0.5 to 1 min. The solution was titrated with 0.1 N NaOH in methanol solution to a faint but permanent pink color. The actual volume of the NaOH used was recorded and the acid value was calculated using the following equation:

Acid Value (in mgKOH/g)=(A−B)×N×56.1/W

Where

A=volume of the NaOH solution used in titrating the sample;

B=volume of the NaOH solution used in titrating the blank;

N=normality of sodium thiosulfate solution; and

W=weight of the sample.

The invention has been described with reference to various specific and preferred embodiments and techniques. It should be noted that the descriptions of the various embodiments provided in this disclosure may be of overlapping scope. The embodiments discussed in this disclosure are merely illustrative and are not meant to limit the scope of the present invention. It should be understood that many variations and modifications may be made while remaining within the spirit and scope of the invention.

What is claimed is:

1. A reactive oil comprising a reactive polyol fatty acid ester stock having a peroxide value of at least 150; an average conjugated unsaturation content of at least 0.02; and a Brookfield viscosity at 25° C. of no more than 1,000 cPs.

2. The reactive oil of claim 1 having an Iodine Value of at least 110.

3. The reactive oil of claim 1 having a peroxide value of at least 200.

4. The reactive oil of claim 1 having an average conjugated unsaturation content of 0.05 to 0.3.

5. The reactive oil of claim 1 having an acid value of no more than 3.0.

6. The reactive oil of claim 1 having of density of 0.935 to 0.955.

7. The reactive oil of claim 1 comprising at least about 80 wt. % of the reactive polyol fatty acid ester stock.

8. The reactive oil of claim 7 further comprising a solvent.

9. The reactive oil of claim 1 comprising a reactive fatty acid ester of a polyol selected from the group consisting of glycerol, ethylene glycol, diethylene glycol, triethylene glycol, neopentylglycol, pentaerythritol, trimethylolethane, trimethylolpropane, sorbitol, cyclohexanediol, inositol, glucose, galactose, sorbose and mixtures thereof.

10. A process for producing a reactive ester stock comprising:
reacting an unsaturated polyol fatty acid ester stock with oxygen at a temperature of no more than 100° C. to form a blown ester product having a peroxide value of at least about 200 and a Brookfield viscosity at 25° C. of no more than about 500 cPs.

11. The process of claim 10 wherein the unsaturated polyol fatty acid ester stock has an Iodine Value of at least about 120.

12. The process of claim 10 wherein the blown ester product has a Brookfield viscosity at 25° C. of no more than about 250 cPs.

13. The process of claim 10 wherein the blown ester product has a peroxide value of at least about 250.

14. The process of claim 10 wherein reacting the unsaturated polyol fatty acid ester stock with the oxygen comprises passing a stream of air through the unsaturated polyol fatty acid ester stock.

15. The process of claim 10 comprising reacting the unsaturated polyol fatty acid ester stock with the oxygen in the presence of a peroxidation catalyst to produce the blown ester product.

16. The process of claim 10 further comprising adding at least about 0.2 wt. % of a reactive ester stock having a peroxide value of at least 150 to the unsaturated polyol fatty acid ester stock before introducing the oxygen.

17. A method for producing a tempering oil comprising:
reacting an unsaturated polyol fatty acid ester stock with to produce a blown ester product having a peroxide value of at least 150 and a Brookfield viscosity at 25° C. of no more than 250 cPs; and
blending the blown ester product with a conjugatively unsaturated polyol fatty acid ester stock to form said tempering oil.

18. The method of claim 17 wherein said tempering oil has an average conjugated unsaturation content of at least about 0.02.

19. The method of claim 17 wherein said tempering oil has a peroxide value of at least 200; a Brookfield viscosity at 25° C. of no more than 100 cPs; and an average conjugated unsaturation content of at least 0.05.

20. A method for producing a treated composite board comprising:
forming a compressible mat including a resin binder and a plurality of fibers, particles, flakes, chips and or a mixture thereof;
applying a tempering oil to at least one side of the compressible mat; and
pressing the compressible mat between a pair of heated plates to form a compressed fiber board;
wherein the tempering oil has a peroxide value of at least about 150; an average conjugated unsaturation content of at least about 0.02; and a Brookfield viscosity at 25° C. of no more than about 250 cPs.

21. The method of claim 20 wherein the tempering oil comprises a drier which includes a polyvalent metal salt.

22. A treated composite substrate produced by a process comprising the steps of:
forming a compressible mat including a resin binder and a plurality of fibers, particles, flakes, chips and or a mixture thereof;
pressing the compressible mat between a pair of heated plates to form a compressed composite substrate;
applying a tempering oil to at least one side of the compressed composite substrate; wherein the tempering oil has a peroxide value of at least about 150; an average conjugated unsaturation content of at least about 0.02; and a Brookfield viscosity at 25° C. of no more than about 250 cPs; and
curing the tempering oil.

23. The method of claim 22 wherein the tempering oil comprises a drier which includes a polyvalent metal salt.

24. A method for producing a tempered fiber board comprising:
forming a compressible mat including a plurality of cellulosic fibers and a resin binder;
pressing the compressible mat between a pair of heated plates to form a compressed fiberboard;
removing the compressed fiberboard from between the heated plates; and
applying a tempering oil to at least one side of the compressed fiberboard before its temperature has dropped below 100° C. to form a treated fiberboard;
wherein the tempering oil has a peroxide value of at least about 150; an average conjugated unsaturation content of at least about 0.02; and a Brookfield viscosity at 25° C. of no more than about 250 cPs.

25. The method of claim 24 further comprising cooling the treated fiberboard to a temperature of no more than 50° C.

26. A reactive polyol fatty acid ester stock having a peroxide value of at least 300; a Brookfield viscosity at 25° C. of no more than 100 cPs; a density of no more than 0.945; and an acid number of no more than 1.5 mgKOH/g.
